(19) United States
(12) Patent Application Publication
(10) Pub. No.: US 2019/0016676 A1
(43) Pub. Date: Jan. 17, 2019

(54) NOVEL HETERO-DIELS-ALDER CROSS-LINKER AND USE THEREOF IN REVERSIBLY CROSSLINKED POLYMER SYSTEMS

(71) Applicant: Evonik Degussa GmbH, Essen (DE)

(72) Inventors: Friedrich Georg SCHMIDT, Haltern am See (DE); Uwe PAULMANN, Luedinghausen (DE); Christian RICHTER, Muenster (DE); Marcel INHESTERN, Recklinghausen (DE); Christian MEIER, Darmstadt (DE); Christopher BARNER-KOWOLLIK, Stutensee (DE); Kai PAHNKE, Karlsruhe (DE); Miguel Angel SANZ, Dortmund (DE); Sumaira UMBREEN, Dortmund (DE); Christian Ewald JANSSEN, Recklinghausen (DE)

(73) Assignee: Evonik Degussa GmbH, Essen (DE)

(21) Appl. No.: 16/065,895

(22) PCT Filed: Jan. 20, 2017
(86) PCT No.: PCT/EP2017/051158

§ 371 Date: Jun. 25, 2018

(30) Foreign Application Priority Data
Jan. 29, 2016 (EP) 16153363.3

(51) Int. Cl. C07C 327/36; C08L 67/06; C08L 33/06; C08L 33/12

(52) U.S. Cl. CPC C07C 327/36; C08L 67/06; C08L 2312/00; C08L 33/12; C08L 33/066

(57) ABSTRACT

The invention relates to a novel hetero-Diels-Alder crosslinker, to a process for the production thereof and to the use thereof for reversibly crosslinking polymer systems.

### FIELD OF THE INVENTION

The invention relates to a novel hetero-Diels-Alder crosslinker, to a process for the production thereof and to the use thereof for reversibly crosslinking polymer systems.

Applications for this crosslinker building block and its derivatives include production of reversibly crosslinking polymer systems for moulding materials for injection moulding and extrusion, production of foams, applications in the field of additive manufacturing, for example by the SLS process or the FDM process, production of composite components by the RTM process, production of storage-stable prepregs and moulded articles or composite components produced therefrom and also adhesives and coatings. The present invention relates to a novel method for reversible crosslinking of adhesive or coating materials for example.

The reversible crosslinking method enables a very rapid crosslinking even at room temperature and a breaking-apart of the crosslinks at higher temperatures so that thermoplastic processability is recovered and, for example, the originally adhesive-bonded substrates may be easily separated from one another again. A particular aspect is that a plurality of cycles of a crosslinking and a breaking-apart of the crosslinks are possible with the present system.

### PRIOR ART

Methods for reversible crosslinking of polymers are of great interest for a broad field of applications. In adhesive bonding applications for example a very wide variety of possibilities for the automobile industry or the semiconductor industry are described. However, such adhesives are also of interest for the construction of machines, high-precision mechanical devices or in the building industry. In addition to adhesive bonding applications, reversibly crosslinkable polymers may also be of interest in sealants, coating materials such as varnishes or paints or in the production of moulded articles.

DE 198 32 629 and DE 199 61 940 describe processes where adhesives based on epoxy, urea, (meth)acrylate or isocyanate are thermally decomposed. The adhesive formulation from DE 199 61 940 further comprises a thermally unstable substance which is activated upon heating. The adhesive layer in DE 198 32 629 is destroyed by the supply of a particularly large amount of energy. Deactivation of the adhesive layer is irreversible in both cases.

US 2005/0159521/US 2009/0090461 describe an adhesive system that is free-radically crosslinked by irradiation with actinic radiation and destroyed by ultrasound. This process too is irreversibly no longer performable after one adhesive bonding cycle.

In EP 2 062 926 the chains of a polyurethane for adhesive bonding applications have thermally labile, sterically hindered urea groups incorporated therein which through supply of thermal energy are destroyed, thus reducing the adhesive action sufficiently to break apart the bond.

US 2009/0280330 describes an adhesive system which can probably be used more than once and has a bilayer construction. One layer is a shape memory layer which may be thermally flexible or cured. The other layer is a dry adhesive which has different adhesive strengths depending on the structure. However, the problem with such a system is the bilayered structure which is complex and costly to construct and the expected residual tackiness after heating of the shape memory layer.

Methods for the construction of block copolymers have been the subject of research for a number of years, especially in academia, under the umbrella term “click chemistry”. Here, two different homopolymers with linkable end groups are combined and, by means of a Diels-Alder reaction, a Diels-Alder-analogous reaction or another cycloaddition for example, joined. The objective of this reaction is the construction of thermally stable, linear and optionally high molecular weight polymer chains. Inglis et al. (Macromolecules 2010, 43, PP.33-36) for example describe for this purpose polymers having cyclopentadienyl end groups which are obtainable from polymers produced by ATRP. These cyclopentadiene groups can react very rapidly in hetero-Diels-Alder reactions with polymers having electron-poor dithioesters as end groups (Inglis et al. Angew. Chem. Int. Ed. 2009, 48, PP. 2411-2414).

The use of monofunctional RAFT polymers for linking with monofunctional polymers having a dihydrothiopyran group via a hetero-Diels-Alder reaction may be found in Sinnwell et al. (Chem. Comm. 2008, 2052-2054). This method allows AB diblock copolymers to be realized. Rapid variants of this hetero-Diels-Alder linking for the synthesis of AB block copolymers with a dithioester group present after a RAFT polymerization and with a dienyl end group are described in Inglis et al. (Angew. Chem. Int. Ed. 2009, 48, PP.2411-14) and in Inglis et al. (Macromol. Rapd Commun. 2009, 30, PP.1792-98). The analogous production of multiarm star polymers is found in Sinnwell et al. (J. Pol.Sci.: Part A: Pol.Chem. 2009, 47, PP.2207-13).

U.S. Pat. No. 6,933,361 describes a system for producing easily-repairable, transparent moldings. The system is composed of two polyfunctional monomers which polymerize by means of a Diels-Alder reaction to form a high-density network. One functionality is a maleimide and the other functionality is a furan. The thermal switching of a high-density network of this kind is used for repair thereof. Crosslinking takes place at temperatures above 100° C. The partial reverse reaction at even higher temperatures.

Syrett et al. (Polym.Chem. 2010, DOI: 10.1039/b9py00316a) describe star polymers for use as flow improvers in oils. These polymers have self-healing properties controllable by means of a reversible Diels-Alder reaction. To this end, monofunctional polymethacrylate arms are combined with polymethacrylates which in the middle of the chain, as a fragment of the initiator used, comprise a group which can be used in a reversible Diels-Alder reaction.

EP 2 536 797 discloses a reversibly crosslinkable system composed of two components A and B. Component A is a compound having at least two dienophilic groups and component B is a compound having at least two diene functionalities. In terms of the maximum number of possible switching cycles and the stability of the compositions in storage, the combinations of components A and B that are disclosed in EP 2 536 797 are certainly amenable to further optimization.

In addition, there is further prior art that is relevant for applications of reversibly crosslinking systems in the technical field of composites in particular. Fibre-reinforced materials in the form of prepregs are already used in many industrial applications because of their ease of handling and the increased efficiency during processing in comparison with the alternative wet-layup technology.

Industrial users of such systems, in addition to faster cycle times and higher storage stabilities—even at room temperature—are also demanding a way of cutting the prepregs to size, without contamination of the cutting tools with the often sticky matrix material in the course of automated cutting-to-size and laying-up of the individual prepreg layers. Various moulding processes, for example the reaction transfer moulding (RTM) process, comprise introducing the reinforcing fibres into a mould, closing the mould, introducing the crosslinkable resin formulation into the mould, and then crosslinking the resin, typically by application of heat.

One of the limitations of such a process is the relative difficulty in laying the reinforcing fibres into the mould. The individual plies of the woven or non-crimp fabric have to be cut to size and conformed to the different geometries of the particular parts of the mould. This can be both time-consuming and complicated, especially when the mouldings are also to contain foam cores or other cores. Premouldable fibre reinforcement systems with easy handling and existing forming options would be desirable here.

As well as polyesters, vinyl esters and epoxy systems there are a series of special resins in the field of crosslinking matrix systems. These also include polyurethane resins which, because of their toughness, damage tolerance and strength, are used particularly for production of composite profiles via pultrusion processes. A disadvantage often mentioned is that the isocyanates used are toxic. However, the toxicity of epoxy systems and the hardener components used there should also be regarded as critical. This is especially true for known sensitizations and allergies.

In addition, most matrix materials to produce prepregs for composites have the disadvantage that at the point of application to the fibre material they are either in solid form, for example in the form of a powder, or in the form of a highly viscous liquid or melt. In either case, the fibre material is only minimally penetrated by the matrix material, and this may in turn lead to suboptimal stability for the prepreg and/or the composite part.

Prepregs and composites produced therefrom that are based on epoxy systems are described, for example, in WO 98/50211, EP 0 309 221, EP 0 297 674, WO 89/04335 and U.S. Pat. No. 4,377,657. WO 2006/043019 describes a process for producing prepregs based on epoxy resin-polyurethane powders. Furthermore, prepregs based on thermoplastics in powder form as a matrix are known.

WO 99/64216 describes prepregs and composites and a method for the production thereof where emulsions comprising polymer particles having sufficiently small dimensions to allow envelopment of individual fibres are used. The polymers of the particles have a viscosity of at least 5000 centipoise and are either thermoplastics or crosslinking polyurethane polymers.

EP 0 590 702 describes powder impregnations for production of prepregs where the powder consists of a mixture of a thermoplastic and a reactive monomer/prepolymer. WO 2005/091715 likewise describes the use of thermoplastics for production of prepregs.

Prepregs produced using Diels-Alder reactions and potentially activatable retro-Diels-Alder reactions are likewise known. A. M. Peterson et al. (ACS Applied Materials & Interfaces (2009), 1(5), 992-5) describe corresponding groups in epoxy systems. This modification bestows self-healing properties on the component parts. Systems which are analogous but do not rely on an epoxy matrix are also found inter alia in J. S. Park et al. (Composite Science and Technology (2010), 70(15), 2154-9) or in A. M. Peterson et al. (ACS Applied Materials & Interfaces (2010), 2(4), 1141-9). However, none of the cited systems makes it possible to post-modify the composites beyond self-healing. The classic Diels-Alder reaction is only insufficiently reversible under the conditions which are possible, so only minimal effects—as may be sufficient for self-healing of damaged component parts—are possible here.

EP 2 174 975 and EP 2 346 935 each describe thermally recyclable composite materials usable as a laminate which incorporate bismaleimide and furan groups. As will be readily apparent to a person skilled in the art, such a system can be reactivated, i.e. decrosslinked to at least a large extent, only at relatively high temperatures. Such temperatures, however, tend rapidly to induce further secondary reactions, and so the mechanism as described is only suitable for recycling but not for modifying the composites.

WO 2013/079286 describes composite materials and prepregs for the production thereof which include groups for a reversible hetero-Diels-Alder reaction. These systems are reversibly crosslinkable and hence the mouldings are even recyclable. However, these systems can only be applied as a liquid 100% system or from an organic solution. This puts distinct limits on the usefulness of this technology.

The systems described are all either based on organic solvents or applied in the form of a melt or in the form of a liquid 100% system. None of the systems described, however, can be applied in the form of an aqueous dispersion. Yet specifically such aqueous systems would have immense advantages in relation to industrial safety and additionally available processing technologies to produce prepregs and/or composite materials.

EP 2 931 817 described the production and use of a novel crosslinker for reversible hetero-Diels-Alder crosslinking. While the crosslinker described therein is notable for a rapid reaction with appropriate dienes it does need to be stabilized with cyclopentadiene or similarly dienes as a protecting group during synthesis. The pure crosslinker is not stable in bulk either.

### PROBLEM

The problem addressed by the present invention is that of providing a novel reversible crosslinking method employable in different applications and in a broad formulation spectrum.

The problem addressed is further that of finding crosslinker structures which have sufficient thermal stability without protecting groups to render the use of cyclopentadiene or similar structures as blocking agents unnecessary.

Furthermore, the synthesis steps and the yields achieved should be improved to provide a simple and robust method of production to ensure economic production of the crosslinking systems and the reactant costs too should be reduced compared to the systems known from the prior art.

In addition, the retro-Diels-Alder reaction should take place at temperatures that permit glass transition temperatures of the overall system of greater than 100° C. and simultaneously the processing temperatures of a system that is for example methacrylate-based need not be increased above 240° C.

Further problems not explicitly mentioned will be apparent from the entirety of the description, claims, and examples which follow.

Solution

The problems were solved by developing a novel formulation suitable for performing a reversible crosslinking mechanism which is employable for various polymers irrespective of the formulation constituents such as binders. It was found that, surprisingly, the stated problems can be solved by a novel formulation that is crosslinkable by means of a hetero-Diels-Alder reaction.

This novel, reversibly crosslinkable formulation which is crosslinkable by means of a hetero-Diels-Alder reaction comprises a component A having at least two dienophile double bonds, wherein component A comprises at least one instance of the following structural unit Z,

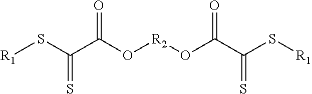

wherein R₁ is an alkyl or alkylene radical having 1 to 20 carbon atoms, wherein the alkylene radical may be bonded to further instances of the structures shown.

The formulation further comprises a component B having at least one diene functionality.

The reversible crosslinking possible with the formulation according to the invention enables a very rapid reaction even at a low first temperature and a breaking-apart of the crosslinks at higher temperatures so that thermoplastic processability is recovered and for example the originally crosslinked layers when employed in the field of individual layers pressed into laminates in composites can be easily separated from one another again or for example the crosslinked individual layers present as prepregs for example can be subjected to forming and pressed into a laminate. A particular aspect is that a plurality of cycles of a crosslinking and a breaking-apart of the crosslinks are possible with the present system.

The described crosslinker/chain extender molecules in pure form are sufficiently stable to temperature Increases not to require blocking with protecting groups.

In particular, the formulations according to the invention have the following particular advantages:
no protecting groups/blocking groups are required for the reactive dienophile in the synthesis.
very simple and robust synthesis with cost-effective reactants and high yield
the formulation is temperature-resistant to above 200° C. even without protecting groups
the retro-hetero-Diels-Alder reaction is effected at temperatures permitting melting point/glass transition temperatures of the overall system of greater than 100° C.

It is preferable when at least one of these two components A or B has more than two functionalities.

It is similarly preferable when at least one of the components A or B is present in the form of a polymer.

It is also preferable when the formulation is crosslinkable at room temperature. This crosslinking can be reversed again to an extent of at least 50% at a higher temperature.

Component A is obtainable by the following general synthetic route for example:

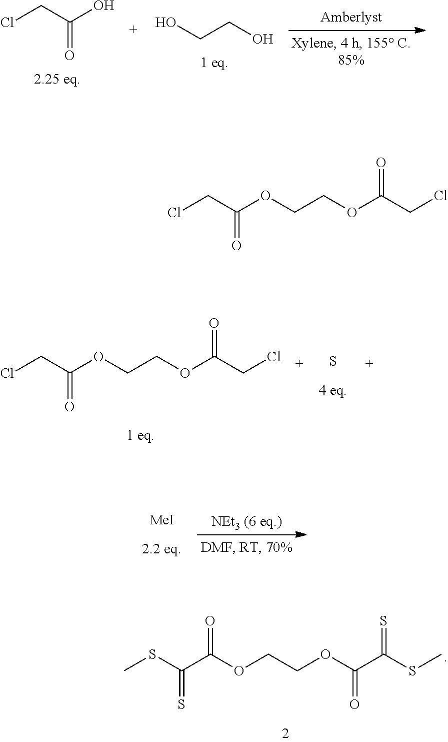

For a longer alkylene chain (R₂) it is also possible to employ other diols, for example hexanediol, in place of the ethylene glycol.

It is particularly preferable when component A is a compound having a plurality of the cited structural units Z. It is especially preferable when the radicals R₁ are alkylene groups having between 1 and 5 carbon atoms by means of which the structural units Z are bonded to one another. R₂ is preferably an alkyl group having 2 to 10 carbon atoms.

It is very particularly preferable when component A is the compound

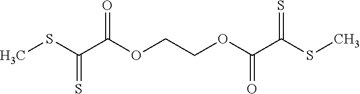

and/or the compound

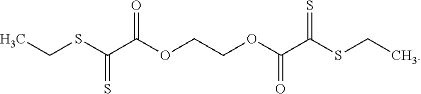 [SMILES: CCSC(=S)C(=O)OCCOC(=O)C(=S)SCC]

In a particular embodiment component B is a polymer. Preferred polymers are polyacrylates, polymethacrylates, polystyrenes, mixed polymers made of acrylates, methacrylates and/or styrenes, polyacrylonitrile, polyethers, polyesters, polylactic acids, polyamides, polyesteramides, polyurethanes, polycarbonates, amorphous or semicrystalline poly-α-olefins, EPDM, EPM, hydrogenated or non-hydrogenated polybutadienes, ABS, SBR, polysiloxanes and/or block, comb and/or star copolymers of these polymers.

In terms of component B one skilled in the art may choose suitable compounds having diene functions suitable for a hetero-Diels-Alder reaction relatively freely. The following three alternatives have proven particularly suitable:

In the first alternative component B is one of the following compounds:

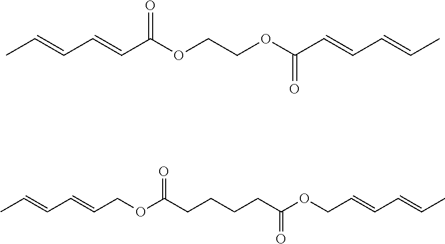

In a second alternative component B is a polymer obtained by copolymerization of at least one of the following comonomers:

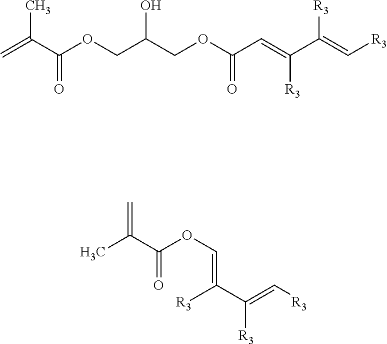

The radicals R₃ may be identical or different radicals. R₃ is preferably hydrogen and/or an alkyl radical having 1 to 10 carbon atoms.

These monomers may be copolymerized with (meth)acrylates and/or styrene for example.

In the third preferred embodiment component B is a polyamide, a polyester or a polycarbonate having at least one diene functionality.

In a particular embodiment of the present invention the formulation according to the invention is not crosslinked but rather a chain extension and thus a switching between two different thermoplastic states is effected. In such a formulation component A has precisely one structural unit Z and component B has precisely one diene group.

The ‘(meth)acrylates’ notation as used in this text is to be understood as meaning alkyl esters of acrylic acid and/or of methacrylic acid.

In a further possible embodiment component B is a bifunctional polymer produced by means of atom transfer radical polymerization (ATRP). In this case functionalization with the diene groups is effected via a substitution of terminal halogen atoms that is polymer-analogous or performed during termination. This substitution may be effected by addition of diene-functionalized mercaptans for example.

A further aspect of the present invention is the process for reversibly crosslinking the formulations according to the invention. When performing this process a formulation composed of at least two different components A and B is crosslinked at room temperature by means of a hetero-Diels-Alder reaction. In a second process step at a higher temperature at least 50%, preferably at least 90% and particularly preferably at least 99%, of the crosslinks are broken apart again by means of a retro-hetero-Diels-Alder reaction.

When performing this second process step at least 90 wt %, preferably at least 95 wt % and particularly preferably at least 98 wt % of the formulation becomes soluble again in a solvent suitable for the formulation before the crosslinking at a temperature above 80° C. preferably within 5 min, at most within 10 min. The previous crosslinking was so extensive that during a 5-minute washing with the same solvent, not more than 5 wt %, preferably not more than 2 wt % and particularly preferably not more than 1 wt % of the formulation could be dissolved. The term “formulation” and all percentages associated therewith in this case relate only to components A and B. Further formulation constituents, such as may be added in a coating or adhesive composition for example are not taken into account in this consideration. In the text below, the expression “formulation” in the context of this specification describes exclusively the components A and B and an optional crosslinking catalyst. The expression “composition” by contrast comprehends not only the formulation but also additionally added components. These additional components may be additive substances selected specifically for the respective application, for example fillers, pigments, additives, compatibilizers, cobinders, plasticizers, impact modifiers, thickeners, defoamers, dispersing additives, rheology improvers, adhesion promoters, scratch-resistance additives, catalysts or stabilizers.

Similarly to the previously described formulation, in the process initially components A and B and also optional further additive substances are brought together.

At room temperature the crosslinking reaction may take place within 10 min, preferably within 5 min, particularly preferably within 2 min and very particularly preferably within one minute. To accelerate the crosslinking a crosslinking catalyst may be added after the mixing of components A and B. These crosslinking catalysts are generally strong acids such as trifluoroacetic acid or sulphuric acid or strong Lewis acids, for example boron trifluoride, zinc dichloride, titanium dichloride diisopropoxide or aluminium trichloride.

In an alternative embodiment, crosslinking may also be accelerated without a catalyst, for example by thermal means. In this case the activation temperature is below the temperature required for the retro-hetero-Diels-Alder reaction.

In a further alternative embodiment, independently of the activation of the crosslinking reaction, the formulation comprises a further catalyst which lowers the activation temperature of the retro-hetero-Diels-Alder reaction. These catalysts may be iron or an iron compound for example.

The formulations and processes of the invention may be employed in a very wide variety of fields of application. The list which follows gives examples of a number of preferred fields of application without limiting the invention in any form whatsoever in this regard. Such preferred fields of application are adhesives, sealants, moulding materials, foams, varnishes, paint, coatings, oil additives—for example flow improvers—or inks.

Examples of moulding materials are for example polyester, polycarbonate, poly(meth)acrylates or polyamide where it is a retro-hetero-Diels-Alder decoupling occurring at elevated temperature combined with the polymer chain linkages reforming at lower temperatures that makes a reduced viscosity/first nascent flowability that is advantageous for plastics injection moulding possible in the first place. The polymer chain linkages which reform at lower temperatures and during cooling improve the mechanical properties of the moulding for example. The moulding compounds may generally be injection-moulding or extrusion moulding materials for example.

When using the similarly inventive formulations for chain extension said formulations thus at a temperature above the retro-hetero-Diels-Alder temperature, i.e. in the “open” state, make it possible, through the lowe viscosity of the melt, a) to better reproduce relatively fine structures, b) to produce parts at lower pressures and/or c) having thinner walls which, however, exhibit the properties of higher molecular weight polymers after the relinking.

When using the formulations according to the invention to achieve crosslinking it is only when said formulations are thus at a temperature above the retro-hetero-Diels-Alder temperature, i.e. in the “open” state, that they may be employed in the processing methods customary for thermoplastic materials of construction, for example blow moulding, injection moulding or extrusion processes, at all. The properties of the crosslinked polymers achievable after the recrosslinking show a markedly enhanced performance such as is familiar to one skilled in the art also in polymers that have been irreversibly crosslinked by radiative crosslinking.

These inks are for example compositions that are applied by thermal means and undergo crosslinking on the substrate. Using conductive oligomers or additives for generating conductivity in general affords an electrically conducting ink which may be processed by bubble jet processes for example.

Examples from the fields of application varnishes, coatings and paint are compositions which are capable of impregnating or wetting for example porous materials particularly readily in the decrosslinked state and as a result of the crosslinking reaction afford highly coherent materials.

Similar characteristics are important for adhesives which should have a high cohesion but are nevertheless intended to easily wet the surfaces of the materials to be adhesive-bonded. A further application in the field of adhesive bonding is for example a joint which is needed only temporarily and is later to be broken apart such as may occur in various production processes, for example in automotive engineering or in mechanical engineering.

Another conceivable application is the adhesive bonding of components which, viewed over the lifetime of the product as a whole, are highly likely to be replaced, and which therefore ought to be removable again as easily as possible and without residue. One example of such an application is the adhesive bonding of car windscreens.

One particular example for adhesives or sealants is use in food packagings which open or can be broken apart automatically during heating, such as in a microwave, for example.

One example of applications in the field of rapid prototyping for the crosslinking and decrosslinking materials described here can be found in the field of FDM (fused deposition modelling), SLS (selective laser sintering) or in 3D printing by ink-jet methods with low-viscosity melts.

The application of the formulations according to the invention in the field of composites is particularly preferred.

Thus the formulations according to the invention may be employed for example as a dispersion for the impregnation of fibre material, for example carbon fibres, glass fibres or polymer fibres. The fibres impregnated in this way may then in turn be used for producing prepregs by known processes.

The invention thus also relates to emulsion polymers for example which are intraparticulately crosslinked with the inventive crosslinker molecules of the formulation by means of the hetero-Diels-Alder mechanism. The crosslinked polymers can then be wholly or partly decrosslinked by thermal processing, for example in the form of a composite matrix, via a retro-hetero-Diels-Alder reaction and interparticulately recrosslinked on cooling. This provides a second route to storage-stable prepregs for composites. But it is also possible to thus realize other materials that have thermoset properties at use temperature but thermoplastic processing properties at a higher temperature.

Use as a matrix material for endless-fibre-reinforced plastics thus affords semi-finished composites having improved processing properties compared to the prior art, which are usable for the production of high-performance composites for a wide variety of different applications in the fields of construction, automotive and aerospace, in the energy industry (for example in wind power plants) and in boat- and shipbuilding. The reactive compositions usable according to the invention are eco-friendly, inexpensive, have good mechanical properties, are simple to process and are characterized by good weather resistance and also by a balanced ratio between hardness and flexibility. In the context of this invention, the term “semi-finished composite” is used synonymously with the terms “prepreg” and “organic sheet”. A prepreg is generally a precursor of thermoset composite components. An organic sheet is normally a corresponding precursor of thermoplastic composite components.

1. A reversibly crosslinkable formulation, crosslinkable by hetero-Diels-Alder reaction, the formulation comprising a component A having at least two dienophile double bonds, and a component B having at least one diene functionality,
wherein:
the component A comprises at least one instance of the following structural unit (Z): 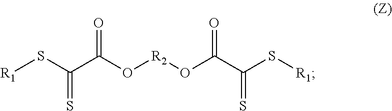
and
R₁ and R₂ are independently an alkyl or alkylene radical having 1 to 20 carbon atoms, in which the alkylene radical may be bonded to further instances of the structural unit (Z).

2. The formulation according to claim 1, wherein:
at least one of the components A or B comprises more than two functionalities;
at least one of the components A or B is present in the form of a polymer, and
the formulation is crosslinkable at room temperature, and the crosslinking is reversible to an extent of at least 50% at a higher temperature.

3. The formulation according to claim 1, wherein:
the component A is a compound having a plurality of the structural unit (Z) and these are bonded to one another with alkylene groups R₁ having between 1 and 5 carbon atoms; and
R₂ has between 2 and 10 carbon atoms.

4. The formulation according to claim 3, wherein the component A is at least one of the following compounds:

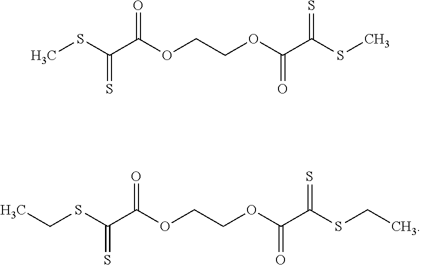

5. The formulation according to claim 1, wherein the component B is a polymer.

6. The formulation according to claim 5, wherein the polymer is selected from the group consisting of polyacrylates, polymethacrylates, polystyrenes, mixed polymers made of acrylates, methacrylates and/or styrenes, polyacrylonitrile, polyethers, polyesters, polylactic acids, polyamides, polyesteramides, polyurethanes, polycarbonates, amorphous or semicrystalline poly-α-olefins, EPDM, EPM, hydrogenated or non-hydrogenated polybutadienes, ABS, SBR, polysiloxanes and block, comb and/or star copolymers of these polymers.

7. The formulation according to claim 1, wherein the component B is one of the following compounds:

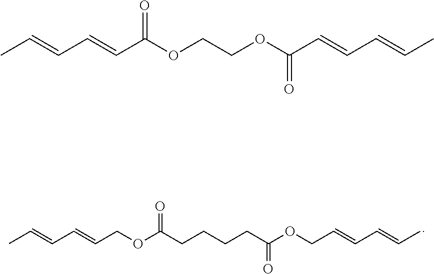

8. The formulation according to claim 1, wherein the component B is a polymer obtained by copolymerizing at least one of the following comonomers: 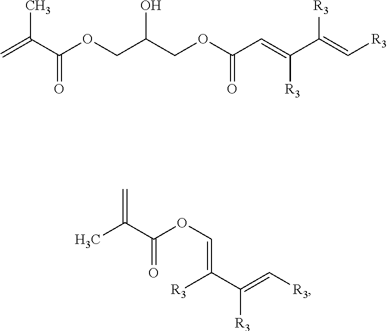
wherein R3 independently represents hydrogen or an alkyl radical having 1 to 10 carbon atoms.

9. The formulation according to claim 1, wherein the component B is a polyamide, a polyester or a polycarbonate having at least one diene functionality.

10. The formulation according to claim 1, wherein the component A has one of the structural unit (Z), and the component B has one diene group.

11. A process for reversible crosslinking, the process comprising crosslinking the formulation of claim 1 at room temperature by a hetero-Diels-Alder reaction, and at a higher temperature breaking at least 50% of the crosslinks by means a retro-hetero-Diels-Alder reaction.

12. The process according to claim 11, wherein at a temperature above 80° C. at least 90% of the formulation is soluble in a solvent suitable for the formulation before the crosslinking.

13. The process according to claim 11, wherein the crosslinking occurs within 2 min after mixing of the components A and B.

14. The process according to claim 11, wherein the crosslinking occurs within 2 min after mixing of the components A and B with a crosslinking catalyst.

15. A composition, comprising the formulation of claim 1, the composition being selected from the group consisting of adhesives, sealants, moulding materials, foams, varnishes, paints, coatings and inks.

16. A composite, comprising the formulation of claim 1, wherein the composite is adapted to function as a composite in the fields of construction, automotive and aerospace, in the energy industry and in boat- or shipbuilding.
